(19) United States
(12) Patent Application Publication
(10) Pub. No.: US 2025/0170428 A1
(43) Pub. Date: May 29, 2025

(54) ULTRASOUND GENERATING DEVICE PROVIDED WITH AUTOMATIC CONTROL FUNCTION FOR ULTRASOUND, SYSTEM, AND SKIN MEASURING DEVICE

(71) Applicant: JEISYS MEDICAL INC., Seoul (KR)

(72) Inventor: Dong Hwan Kang, Seoul (KR)

(73) Assignee: JEISYS MEDICAL INC., Seoul (KR)

(21) Appl. No.: 19/038,227

(22) Filed: Jan. 27, 2025

Related U.S. Application Data
(63) Continuation of application No. PCT/KR2023/006283, filed on May 9, 2023.

(30) Foreign Application Priority Data
Jul. 28, 2022 (KR) 10-2022-0093774

(51) Int. Cl. A61N 7/00

(52) U.S. Cl. CPC A61N 7/00; A61N 2007/0034

(57) ABSTRACT

An ultrasound generating device includes: an ultrasound generator that irradiates ultrasonic waves to the skin; a receiver that receives, from a skin measuring device, depth information about a skin layer of each of a plurality of regions; a memory which stores the received depth information, location information for each of the plurality of regions, and mapping information mapping a preset treatment condition to each of the plurality of regions; and a processor that controls the ultrasound generating unit so as to irradiate, to the skin layer of a corresponding region, ultrasonic waves at at least one of a treatment depth and a treatment intensity corresponding to a corresponding treatment condition on the basis of the mapping information.

### CROSS-REFERENCE TO RELATED APPLICATIONS

The present application is a continuation of International Patent Application No. PCT/KR2023/006283, filed on May 9, 2023, which claims the benefit of priority to Korean Patent Application No. 10-2022-0093774 filed on Jul. 28, 2022. The disclosures of the above-listed applications are hereby incorporated by reference herein in their entirety.

### FIELD

The present disclosure relates to an ultrasound generating device provided with automatic control function for ultrasound, a system, and a skin measuring device.

### BACKGROUND

Ultrasound refers to waves with a frequency of 20 kHz or higher, and has the property of penetrating water, so it is widely used in the medical field, such as ultrasound diagnostic devices and ultrasound treatment devices.

An application of ultrasound in the medical field is an ultrasound imaging device that utilizes the properties of ultrasound penetration and reflection. For example, there is a device that visualizes the time and intensity of reflection as ultrasound penetrates the human body and passes through each organ, thereby obtaining a cross-sectional image of the human body.

In addition, there is a device that uses the heat generated by high-intensity focused ultrasound (HIFU) to burn and remove specific subcutaneous tissues, such as tumors in the skin, or to induce degeneration and regeneration of skin tissue, resulting in skin beauty or skin plastic surgery effects, such as wrinkle improvement.

However, conventional ultrasound generating devices are unable to accurately irradiate ultrasound to an irradiation position during the procedure, which reduces the accuracy of treatment.

In addition, conventional ultrasound generating devices have limitations in shortening the treatment time and maximizing the effectiveness of the treatment because a doctor has to perform the treatment carefully.

### SUMMARY

In an exemplary embodiment, the present disclosure provides an ultrasound generating device having an automatic ultrasound control function. The device includes: an ultrasonic generator configured to irradiate ultrasound to the skin; a receiver configured to receive depth information for each of a plurality of skin regions from a skin measuring device, wherein the depth information for a respective skin region includes a plurality of depths corresponding to a plurality of skin layers for the respective skin region; a memory configured to store mapping information, wherein the mapping information includes a correspondence between (A) a treatment condition and (B) position information of one or more skin regions to be treated for the treatment condition and depth information for each of the one or more skin regions to be treated for the treatment condition; and a processor configured to control the ultrasonic generator based on the mapping information to irradiate ultrasound to each of the one or more skin regions to be treated for the treatment condition at respective depth(s) corresponding to the depth information.

### BRIEF DESCRIPTION OF THE FIGURES

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a diagram illustrating a configuration of an ultrasound generating system to which an ultrasound generating device according to the present disclosure is applied.

FIG. 2 is a diagram illustrating a configuration of the ultrasound generating device and the skin measuring device of FIG. 1.

FIG. 3 is a diagram illustrating an example of a process of capturing an image through the first measurement module of FIG. 2 and measuring a depth of a skin layer for each of a plurality of regions.

FIG. 4 is a diagram illustrating an example of a process of measuring a position for each of a plurality of regions through the second measurement module of FIG. 2.

FIG. 5 is a diagram illustrating an example of a process of storing treatment depth data and ultrasound intensity data of the ultrasonic generator in the memory of FIG. 2.

FIG. 6 is a diagram illustrating an example of a process of measuring a position of a handpiece through the second measurement module of FIG. 2.

FIGS. 7 and 8 are diagrams illustrating an example of a process in which the treatment depth and intensity of the ultrasonic generator are controlled and output using the treatment depth data and the ultrasound intensity data of FIG. 5.

FIGS. 9 and 10 are flowcharts illustrating an example of a method for automatically adjusting ultrasound according to a depth of a skin treatment region of an ultrasound generating device according to the present disclosure.

FIG. 11 is a diagram illustrating an example of displaying a treatment completion state according to a treatment depth at a current irradiation position corresponding to the epidermis as a UI through the notification module of FIG. 2.

FIG. 12 is a diagram illustrating an example of displaying a treatment completion state according to a treatment depth at a current irradiation position corresponding to the dermis as a UI through the notification module of FIG. 2.

### DETAILED DESCRIPTION

Exemplary embodiments of the present disclosure provide for accurately irradiating ultrasound to an irradiation position of ultrasound, thereby improving the accuracy of the treatment.

In addition, exemplary embodiments of the present disclosure provide for maximizing the effect of the treatment while reducing the treatment time, since ultrasound can be irradiated to each part by adjusting the optimal condition in a single treatment.

Technical problems addressed by exemplary embodiments of the present disclosure are not limited to the technical problems mentioned above, and other advantages not mentioned will be clearly understood by those skilled in the art from the following description.

In an aspect of the present disclosure, an ultrasound generating device may include an ultrasonic generator configured to irradiate ultrasound to the skin; a communication module configured to receive depth information for a skin layer of each of a plurality of regions from a skin measuring device; a memory configured to store mapping information that maps each of the received depth information, position information of each of the plurality of regions, and the plurality of regions to a preset treatment condition; and a processor configured to control the ultrasonic generator so that ultrasound is irradiated to the skin layer of the corresponding region with at least one of the treatment depth or intensity corresponding to the treatment condition based on the mapping information.

Furthermore, the depth information may be obtained through a first measurement module of the skin measuring device, and the position information may be obtained through a second measurement module.

Furthermore, the position information may be obtained through at least one of a navigation sensor, a position recognition camera, or an artificial intelligence (AI) camera of the second measurement module.

Furthermore, based on the second measurement module being the AI camera, the position information may further include image information including the plurality of regions on the skin, and wherein the memory may be further configured to store the mapping information that maps each of the depth information, the position information including the image information, and the plurality of regions to the preset treatment condition.

Furthermore, the second measurement module may operate in a state where a patient is fixed so that there is no movement of the patient.

Furthermore, the communication module may be further configured to receive an angle of the skin measuring device, and the angle may be obtained through the first angle measurement module of the skin measuring device.

Furthermore, the second measurement module may be further configured to measure a position of the ultrasonic generator, the processor may be further configured to check the mapping information corresponding to position of handpiece, and the processor may be further configured to control the ultrasonic generator so that the ultrasound is irradiated to the skin layer of the corresponding region with at least with one of the treatment depth or intensity corresponding to the corresponding treatment condition based on the mapping information.

Furthermore, the device may further include a second angle measurement module configured to measure an angle of the ultrasonic generator, and the processor may be further configured to determine whether the angle information measured by the first angle measurement module and the angle information measured by the second angle measurement module match.

Furthermore, the processor may be further configured to output a treatment completion state through a notification module when the treatment is completed on the skin layer of the corresponding region under the corresponding treatment condition.

Furthermore, the communication module may be further configured to receive an image before the procedure and after the procedure for each of the plurality of regions, and the processor may be further configured to transmit data for the image data before the procedure, the image data after the procedure, and the data for the mapping information to the server.

Furthermore, in another aspect of the present disclosure, a skin measuring device for an ultrasound generator may include a first measurement module configured to measure a depth of a skin layer and transmit the depth to the ultrasound generator to map a preset treatment condition to depth information for each of a plurality of regions.

Furthermore, the device may further include a first angle measurement module configured to measure an angle of the skin measuring device and transmit the angle to the ultrasound generator.

Furthermore, in still another aspect of the present disclosure, an ultrasound generation system provided with automatic control function of ultrasound for each treatment region depth of a skin may include a skin measuring device configured to measure a depth of a skin layer for each of a plurality of regions; and an ultrasonic generator configured to irradiate ultrasound to the skin layer of the corresponding region with at least one of a treatment depth or intensity corresponding to a treatment condition based on mapping information that maps each of depth information, position information of each of the plurality of regions, and the plurality of regions to a preset treatment condition.

Furthermore, the ultrasonic generator may include an ultrasonic generator configured to irradiate ultrasound to the skin; a communication module configured to receive depth information for a skin layer of each of a plurality of regions from an external skin measuring device; a memory configured to store mapping information that maps each of the received depth information, position information of each of the plurality of regions, and the plurality of regions to a preset treatment condition; and a processor configured to control the ultrasonic generator so that ultrasound is irradiated to the skin layer of the corresponding region with at least one of the treatment depth or intensity corresponding to the treatment condition based on the mapping information.

Furthermore, a computer program stored in a computer-readable recording medium for executing the present disclosure may be further provided.

Furthermore, a computer-readable recording medium recording a computer program for executing a method for executing the present disclosure may be further provided.

In the drawings, the same reference numeral refers to the same element. This disclosure does not describe all elements of embodiments, and general contents in the technical field to which the present disclosure belongs or repeated contents of the embodiments may be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network. Furthermore, when a certain part “includes” a certain element, other elements are not excluded unless explicitly described otherwise, and other elements may in fact be included.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

First, high-intensity focused ultrasound (HIFU) technology is the latest thermal ablation treatment that burns specific subcutaneous tissues such as tumors in the skin by using the heat generated when high-intensity ultrasound is focused on one point in the skin. This is similar to the principle of focusing warm sunlight with a magnifying glass to light a fire. Since ultrasound easily passes through body tissues, HIFU treatment is performed in a completely non-invasive manner without a knife or even a needle. In other words, by simply pressing the patient's skin on the ultrasound generation surface, specific subcutaneous tissues such as tumors are burned and treated. In addition, HIFU treatment is currently being used to treat uterine fibroids, bone metastases, prostate cancer, breast cancer, pancreatic cancer, liver cancer, and kidney cancer.

This high-intensity focused ultrasound technology may be implemented through an ultrasound generation device. An ultrasound generation device can irradiate ultrasound to the surface of a patient's skin.

The controller of the ultrasound generating device according to the present disclosure in this specification includes various devices that may perform computational processing and provide results to a user. For example, the controller of the ultrasound generating device according to the present disclosure may include a computer, a server device, and a portable terminal, or may be in the form of one of them.

Here, the computer may include, for example, a notebook, a desktop, a laptop, a tablet personal computer (PC), a slate PC, and the like mounted with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

The ultrasound generating device provided with automatic control function for ultrasound for each treatment region depth of a skin according to the present disclosure may control the ultrasound generator so that ultrasound is irradiated to a skin layer of the corresponding region with at least one of a depth or intensity corresponding to the corresponding treatment condition based on the mapping information that maps each of the received depth information, position information of each of the plurality of regions, and the plurality of regions to a preset treatment condition when the ultrasound generator irradiates ultrasound to each of the plurality of regions.

The ultrasound generating device may accurately irradiate ultrasound to the irradiation position of the ultrasound, thereby improving the accuracy of the treatment. In addition, the ultrasound generating device may irradiate ultrasound by adjusting an optimal condition for each region in one treatment process, thereby reducing the treatment time and maximizing the treatment effect.

Hereinafter, the ultrasound generating device provided with automatic control function for ultrasound for each treatment region depth of a skin will be examined in detail.

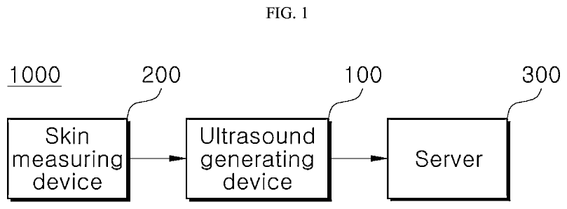

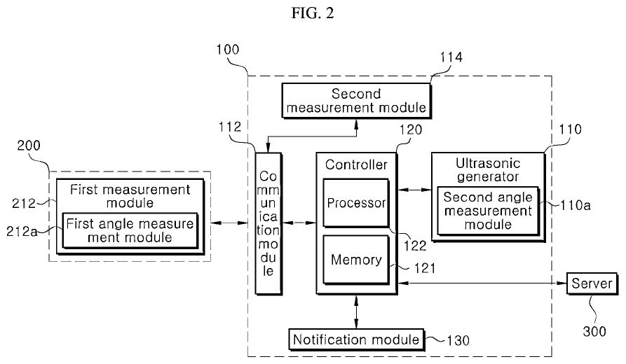

FIG. 1 is a diagram illustrating a configuration of an ultrasound generating system to which the ultrasound generating device according to the present disclosure is applied. FIG. 2 is a diagram illustrating a configuration of the ultrasound generating device and the skin measuring device of FIG. 1.

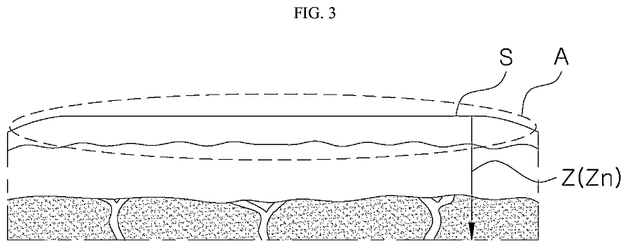

FIG. 3 is a diagram illustrating an example of a process of capturing an image through the first measurement module of FIG. 2 and measuring a depth of a skin layer for each of a plurality of regions.

Referring to FIGS. 1 to 3, an ultrasound generating system 1000 may include an ultrasound generating device 100 and a skin measuring device 200.

The skin measuring device 200 may measure a depth of a skin layer for each of a plurality of regions A. The skin measuring device 200 may include a first measurement module 212.

The first measurement module 212 may measure the depth of the skin layer and transmit it to the ultrasound generating device 100 to map preset treatment conditions to depth information for the skin layer for each of the plurality of regions A. The first measurement module 212 may measure a depth Z of the skin layer for each of the plurality of regions A. For example, the first measurement module 212 may be a medical ultrasound sensor. Meanwhile, the first measurement module 212 may be at least one of a medical ultrasound probe or a medical ultrasound measuring device, and is not limited to a device that measures skin depth. In addition, the skin measuring device 200 may measure the depth of a boundary surface of epidermis, dermis, fat, muscle, superficial musculoaponeurotic system (SMAS), and the like of the skin S. The first measurement module 212 may measure the depth Z of each skin layer corresponding to each irradiation position for each of the plurality of regions A.

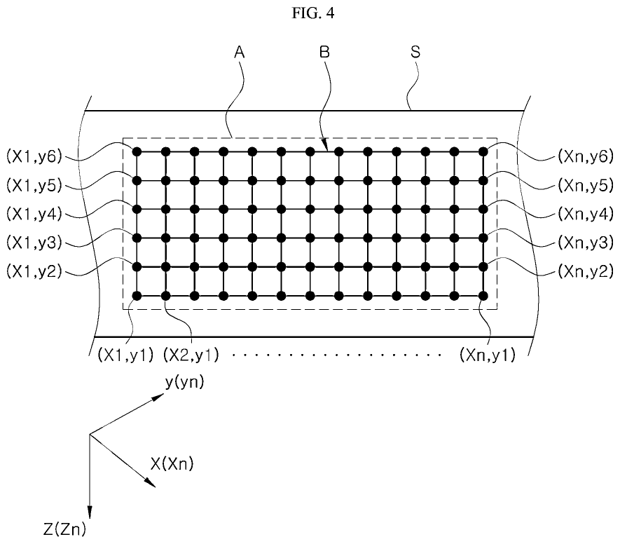

FIG. 4 is a diagram illustrating an example of a process of measuring a position for each of a plurality of regions through the second measurement module of FIG. 2.

Referring to FIG. 4, a second measurement module 114 may be operated in a state where a patient is fixed so that there is no movement of the patient. The second measurement module 114 may measure a position of each of the plurality of regions A to map the treatment condition to the position information of each of the plurality of regions A. The second measurement module 114 may also measure a direction of each of the plurality of regions A. The second measurement module 114 may measure position information B for each of the plurality of regions A using a navigation sensor. The navigation sensor may measure the overall position coordinate values (xn, yn) for the plurality of regions A. Here, xn may be n position coordinate values for the x-axis, and yn may be n position coordinate values for the y-axis.

For example, the navigation sensor may be provided inside the second measurement module 114, and a receiving sensor that receives the position of the navigation sensor may be provided outside the second measurement module 114. An AI camera may recognize whether a specific region of the patient's face is being scanned by reading the image. The position information B further includes image information including the plurality of regions A on the skin S, and the memory 121 may further store the depth information, the position information B including image information, and the mapping information that maps preset treatment condition to each of the plurality of regions A. The communication module 112 may obtain the position information B of which a region of the face is being scanned through the receiving sensor that receives the position of the navigation sensor provided inside the second measurement module 114 or the navigation sensor provided outside the second measurement module 114.

In addition, the second measurement module 114 may measure the position information B for each of the plurality of regions A using a position recognition camera. For example, the position recognition camera may be a 3D camera, and the 3D camera may measure the overall 3D position coordinate values (xn, yn, and Zn) for the plurality of regions A. Here, xn may be n position coordinate values for the x-axis, yn may be n position coordinate values for the y-axis, and Zn may be n position coordinate values for the Z-axis. The 3D camera may also measure position coordinate value for the depth Z of the skin layer.

The second measurement module 114 may scan a region of the face using the navigation sensor, and may scan a region of the face using the position recognition camera. In addition, the second measurement module 114 may increase the accuracy and discrimination power when scanning the face by using the navigation sensor and the position recognition camera together. Meanwhile, a fixing device may be further provided to fix a head so that the patient's face does not move when scanning the face.

In this way, the second measurement module 114 may measure the position information B for the plurality of regions A using at least one of the navigation sensor, the position recognition camera, or the AI camera. The position information B measured through at least one of the navigation sensor, the position recognition camera, and the AI camera may be transmitted to the communication module 112.

Meanwhile, the first measurement module 212 may further include a first angle measurement module 212a. The first angle measurement module 212a may measure an angle of the first measurement module 212 and transmit it to the ultrasound generating device 100. Since the position and depth at which the skin S is measured vary depending on the angle of the first measurement module 212, the first angle measurement module 212a may be provided to accurately measure the skin S. The controller 120 of the ultrasound generation device 100 may receive the angle information of the first measurement module 212 through the communication module 112 and control the ultrasonic generator 110 to generate ultrasound based on the received angle information of the first measurement module 212. For example, the first angle measurement module 212a may be an angle sensor.

The ultrasound generating device 100 may irradiate ultrasound with at least one of the treatment depth or intensity corresponding to the treatment condition to the skin layer of the corresponding region based on the mapping information that maps each of the depth information for the skin layer of each of the plurality of regions A received from the skin measuring device 200, the position information of each of the plurality of regions A, and the plurality of regions A to a preset treatment condition.

The communication module 112 may receive the depth information for the skin layer of each of the plurality of regions A and the position information B for each of the plurality of regions A from the external skin measuring device 200. The depth information is obtained through the first measurement module 212, and the position information is obtained through the navigation sensor, the position recognition camera, or the AI camera of the second measurement module 114.

Here, the communication module 112 may receive and obtain the depth information and the position information B while connected to the external skin measuring device 200 through wired or wireless communication, and may also receive and obtain an angle. The communication module 112 may receive and obtain the depth information and the position information B from a separate device or another server, and may also receive and obtain the angle.

The communication module 112 may include a wired communication module or a wireless communication module that performs wired or wireless communication with the external skin measuring device 200.

The wired communication module may include various wired communication modules such as a local area network (LAN) module, a wide area network (WAN) module, or a value-added network (VAN) module, as well as various cable communication modules such as a Universal Serial Bus (USB), a High Definition Multimedia Interface (HDMI), a Digital Visual Interface (DVI), RS-232 (recommended standard232), power line communication, or a plain old telephone service (POTS).

The wireless communication module may include a wireless communication module that supports various wireless communication methods such as a WiFi module, a Wibro (wireless broadband) module, and a GSM (Global System for Mobile Communication), CDMA (code-division multiple access), WCDMA (Wideband Code Division Multiple Access), UMTS (universal mobile telecommunications system), TDMA (time-division multiple access), LTE (Long Term Evolution), 4G, 5G, and 6G.

The controller 120 may be implemented with a memory 122 that stores data on an algorithm for controlling the operation of components within the device or a program that reproduces the algorithm, and at least one processor 121 that performs the operation using the data stored in the memory 122. Here, the memory 122 and the processor 121 may be implemented as separate chips, respectively. In addition, the memory 122 and the processor 121 may be implemented as a single chip.

The memory 122 may store data supporting various functions of the device, programs for the operation of the controller, may store input/output data, a plurality of application programs (or applications) executed on the device, data for the operation of the device, and commands. At least some of these application programs may be downloaded from an external server via wireless communication.

The memory 122 may include at least one type of storage medium among a flash memory type, a hard disk type, an SSD type (solid-state drive type), an SDD type (Silicon Disk Drive type), a multimedia card micro type, a card type memory (for example, an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, or an optical disk. In addition, the memory 122 may be a database that is separate from the device but connected by wire or wirelessly.

The memory 121 may store the mapping information that maps each of an image, the depth information on the skin layer, the position information B of each of the plurality of regions A, and the plurality of regions A to a preset treatment condition.

As illustrated in FIG. 4, the memory 121 may store the mapping information that maps the entire position coordinate values (xn, yn) corresponding to each irradiation position for each of the plurality of regions A, depth values zn of the entire skin layer for the entire position coordinate values (xn, yn), and the treatment conditions. The processor 122 may map the entire position coordinate values (xn, yn) corresponding to each irradiation position for each of the plurality of regions A, the depth values zn of the entire skin layer for the entire position coordinate values (xn, yn), and the treatment condition.

For example, the memory 121 may store the mapping information in which the first position coordinate value (x1, y1) corresponding to each irradiation position, the depth value z1 of the skin layer for the first position coordinate value (x1, y1), and the treatment condition at the first position coordinate are mapped. In addition, the memory 121 may store the mapping information in which the second position coordinate value (x2, y1) corresponding to each irradiation position, the depth value z2 of the skin layer for the second position coordinate value x2, y1, and the treatment condition at the second position coordinate are mapped.

In this way, the memory 121 may store the mapping information in which the entire position coordinate value (xn, yn) corresponding to each irradiation position for the plurality of regions A, the depth value (zn) of the entire skin layer for the entire position coordinate value (xn, yn), and the treatment condition at the entire position coordinate are mapped, respectively.

For example, the treatment condition is for the treatment purpose, and may be for increasing fat, may be for reducing fat, may be for adding elasticity to the dermis or improving skin texture, may be for treating wrinkles or acne, may be for reducing pain, may be for lifting the jaw line in the case that fat from the cheeks is removed, may be for lifting or tightening the skin. In addition, the treatment purpose may be for treating by the region for skin improvement.

For another example, the treatment condition is for the target irradiation condition of ultrasound according to the treatment depth of each region, and may be for irradiating ultrasound with a specific temperature and specific energy at the treatment depth of the forehead, lower jaw, nasolabial folds, around the eyes, nose, and upper arms, such as the nipple layer, upper dermis, lower dermis, upper fat, fat layer, SMAS, and fascia. In addition, the target irradiation condition of ultrasound according to the treatment depth of each region may be to irradiate ultrasound as the target condition while varying the treatment depth of each region for skin improvement.

Therefore, the memory 121 may store each of the mapping information in which the entire position coordinate value (xn, yn) corresponding to each irradiation position for the plurality of regions A, the depth value (zn) of the entire skin layer for the entire position coordinate value (xn, yn), and the treatment condition at the entire position coordinate are mapped, respectively.

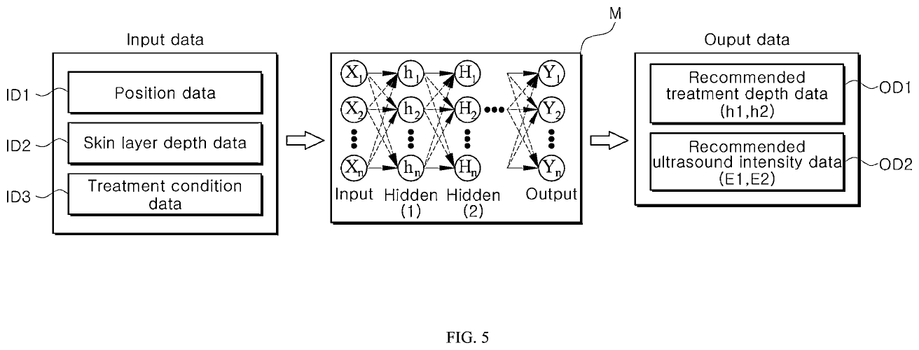

FIG. 5 is a diagram illustrating an example of a process of storing the treatment depth data and ultrasound intensity data of the ultrasonic generator in the memory of FIG. 2.

Referring to FIG. 5, the server 300 electrically connected to the controller 120 may output treatment depth data OD1 and ultrasound intensity data OD2 of the ultrasonic generator 110, which are learned and recommended based on the metadata, position data ID1, skin layer depth data ID2, and treatment condition data ID3.

The treatment recommendation model M may be constructed to learn through the correlation between the position data ID1, the skin layer depth data ID2, and the treatment condition data ID3 included in the input data. The server 300 may transmit the recommended treatment depth data OD1 and ultrasound intensity data OD2 of the ultrasonic generator 110 to the memory 121 of the controller 120. The memory 121 may store the received treatment depth data OD1 and ultrasound energy data OD2 of the ultrasonic generator 110.

Here, the position data ID1 may be the entire position coordinate value (xn, yn) corresponding to each irradiation position for the plurality of regions A, and the depth data ID2 of the skin layer may be the entire skin layer depth value (zn) for the entire position coordinate value (xn, yn).

In addition, the treatment condition data ID3 may include data for each treatment condition in the entire position coordinate, and may include data for the purpose of the treatment. For example, the data for the purpose of the treatment may be data for increasing fat, data for reducing fat, data for adding elasticity to the dermis or improving skin texture, data for treating wrinkles or acne, data for reducing pain, data for lifting the jawline in the case that fat on the cheek is removed, data for lifting or tightening the skin, and the like.

In addition, the treatment condition data ID3 may include data for the target irradiation condition of ultrasound according to the treatment depth of each region as data for each treatment condition in the overall position coordinates. For example, the data for the target irradiation condition of ultrasound according to the treatment depth of each region may be data for applying a specific temperature and specific energy to treatment regions such as the cheek, forehead, lower jaw, nasolabial folds, around the eyes, nose, and upper arms at treatment depths such as the nipple layer, upper dermis, lower dermis, upper fat, fat layer, SMAS, and fascia.

The processor 122 may control the ultrasonic generator 110 so that ultrasound is irradiated to the skin layer of the corresponding region A with the treatment depth and intensity corresponding to the treatment condition when the ultrasonic generator 110 irradiates ultrasound to each of the plurality of regions A based on the mapping information. The processor 122 may adjust at least one of the treatment depth or the intensity of ultrasound corresponding to the corresponding treatment condition to the skin layer of the corresponding region A based on the treatment depth data and the ultrasound intensity data of the ultrasonic generator 110 stored in the memory 121. The ultrasonic generator 110 may irradiate at least one of the adjusted treatment depth or intensity of ultrasound corresponding to the corresponding treatment condition to the skin layer of the corresponding region A.

The second angle measurement module 110a may measure the angle of the ultrasonic generator 110. The processor 122 may determine whether the angle information measured by the first angle measurement module 212a and the angle information measured by the second angle measurement module 110a match. For example, the second angle measurement module 110a may be an angle sensor. The processor 122 may control at least one of the angle or treatment direction of the ultrasonic generator 110 so that ultrasound is accurately irradiated to the skin layer of the corresponding region A with at least one of the treatment depth or intensity corresponding to the corresponding treatment condition based on at least one of the angle information or the treatment direction information stored in the memory 121.

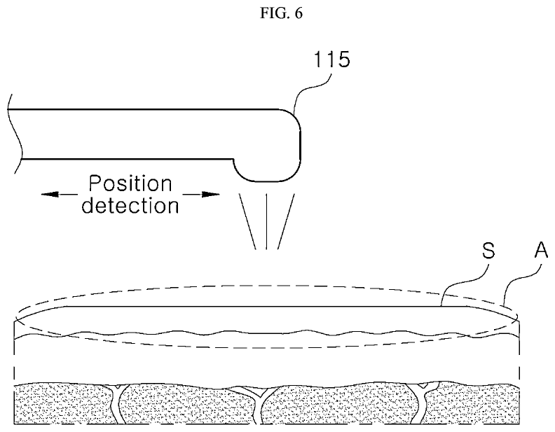

FIG. 6 is a diagram illustrating an example of a process of measuring a position of a handpiece through the second measurement module of FIG. 2.

Referring to FIG. 6, the second measurement module 114 may further measure the position of the ultrasonic generator 110. In addition, the second measurement module 114 may also measure the position of a handpiece 115 coupled with a cartridge housing in which the ultrasonic generator 110 is provided. The second measurement module 114 may be provided inside or outside the handpiece 115 and may measure the position of the handpiece 115 in real time. In addition, the second measurement module 114 may be provided inside or outside the cartridge housing and may measure the position of the handpiece 115 in real time. For example, the second measurement module 114 may include a camera, a position sensor, and the like.

The processor 122 may receive the position data of the handpiece 115 measured through the second measurement module 114 in real time. When the ultrasonic generator 110 irradiates ultrasound for each of the plurality of regions A, the processor 122 may check the mapping information corresponding to the position of the handpiece 115 and control the ultrasonic generator 110 so that ultrasound is irradiated to the skin layer of the corresponding region A with at least one of the treatment depth or intensity corresponding to the corresponding treatment condition based on the mapping information. The processor 122 may adjust at least one of the ultrasound treatments corresponding to the corresponding treatment condition to the skin layer of the corresponding region A based on the treatment depth data and ultrasound intensity data of the ultrasonic generator 110 stored in the memory 121. The ultrasonic generator 110 may irradiate to the skin layer of the corresponding region A with at least one of the treatment depth or intensity of the adjusted ultrasound corresponding to the corresponding treatment condition.

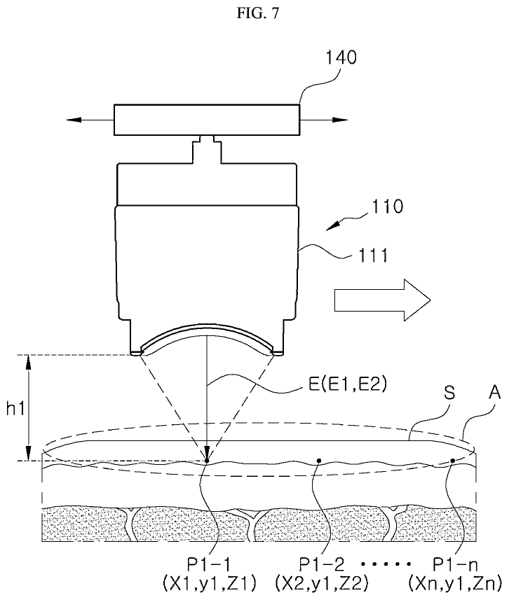

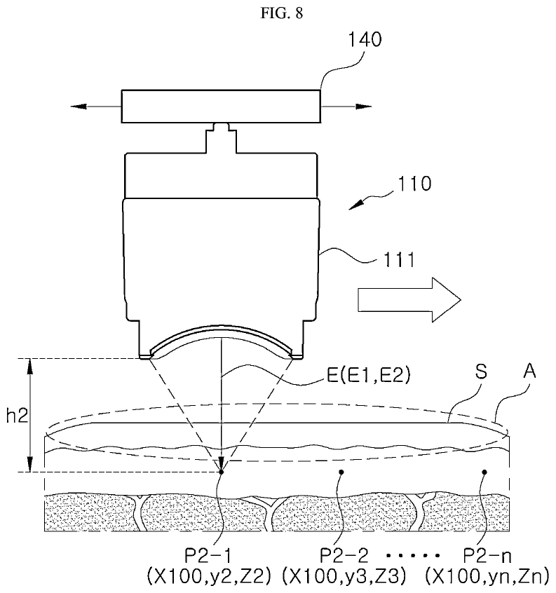

FIGS. 7 and 8 are diagrams illustrating an example of a process in which the treatment depth and intensity of the ultrasonic generator are controlled and output using the treatment depth data and the ultrasound intensity data of FIG. 5.

Referring to FIG. 7, a transfer unit 140 is provided to support the ultrasonic generator 110 by moving the ultrasonic generator 110 to the left or right, or moving it up and down to control at least one of the treatment depth or intensity of the ultrasonic generator 110. The transfer unit 140 may move the ultrasonic generator 110 to the left or right under the control of the controller 120. In the case that the ultrasonic generator 110 sequentially irradiates ultrasound to the irradiation positions P1-1 to P1-n according to the first region corresponding to coordinate values [(x1, y1), (x2, y1), . . . (xn, y1)] of the corresponding region A and the skin depth values (z1, z2, . . . , zn) by the movement of the transfer unit 140, the ultrasonic generator 110 may irradiate ultrasound with at least one of the automatically adjusted treatment depth h1 corresponding to the treatment depth data OD1 or the automatically adjusted intensities E, E1, and E2 corresponding to the ultrasound intensity data OD2. Here, ‘E’ may be ultrasound having the same intensity, and E1 and E2 may be ultrasound having different intensities. The ultrasonic generator 110 may irradiate ultrasound with the same intensity E to the irradiation positions P1-1 to P1-n according to the first region, and may irradiate ultrasound with different intensities E1 and E2. For example, in the case that the first region is a ball, and the ultrasonic generator 110 sequentially irradiates ultrasound to the irradiation positions P1-1 to P1-n of the ball to remove fat from the ball, the ultrasonic generator 110 may irradiate ultrasound to the irradiation positions P1-1 to P1-n of the ball with the same intensity E, and may irradiate ultrasound with different intensities E1 and E2. Here, the present disclosure is not limited to the different intensities being determined only by E1 and E2, and may be set to be finely adjusted according to the skin boundary line of each person.

Referring to FIG. 8, in the case that the ultrasonic generator 110 passes the irradiation positions P1-1 to P1-n according to the first region by the movement of the transfer unit 140, and then sequentially irradiating ultrasound to the irradiation positions P2-1 to P2-n according to the second region corresponding to the position coordinate values [(x100, y2), (x100, y3), . . . , (x100, yn)] and the skin depth values z2, z3, . . . , zn of the corresponding region A, the ultrasonic generator 110 may irradiate ultrasound with at least one of the automatically adjusted treatment depth h2 corresponding to the treatment depth data OD1 and the automatically adjusted intensities E, E1, and E2 corresponding to the ultrasound intensity data OD2. Here, ‘E’ may be ultrasound having the same intensity, and E1 and E2 may be ultrasound having different intensities. The ultrasonic generator 110 may irradiate ultrasound with the same intensity E to the irradiation positions P2-1 to P2-n according to the second region, and may irradiate ultrasound with different intensities E1 and E2. For example, the second region is the jaw border, and when the ultrasonic generator 110 sequentially irradiates ultrasound to the irradiation positions P2-1 to P2-n of the jaw border to lift the jaw border in the case that the fat of the cheek is removed, ultrasound may be irradiated to the irradiation positions P2-1 to P2-n of the jaw border with the same intensity E, and ultrasound may be irradiated with different intensities E1 and E2. The treatment depth of h2 may be deeper than the treatment depth of h1. Here, the present disclosure is not limited to the treatment depth being determined only by h1 and h2, and may be set to be finely adjusted for each person according to the skin border.

Meanwhile, for convenience of explanation, the ultrasonic generator 110 is illustrated as irradiating the irradiation position P1-1 to P1-n according to the first region with a treatment depth of h1, and the irradiation position P2-1 to P2-n according to the second region with a treatment depth of h2, but the ultrasonic generator 110 may be set to irradiate the irradiation position according to the third region with a treatment depth of h3.

Meanwhile, the processor 122 according to the present disclosure may be set to finely adjust at least one of the treatment depth and intensity within the corresponding region A, since the skin layers (i.e., P1-1 to P1-n) may be formed differently for each person within the corresponding region A.

In this way, in the case that the ultrasonic generator 110 irradiates ultrasound to each irradiation position corresponding to the overall position coordinate value (xn, yn) and the overall skin depth value (zn) for each region A, the ultrasound generator 110 may irradiate ultrasound based on the treatment depth data OD1 and ultrasound intensity data OD2 of the ultrasonic generator 110 learned through the correlation between the position data ID1, the depth data ID2 of the skin layer, and the treatment condition data ID3, at least one of the automatically adjusted treatment depth h1 and h2 corresponding to the treatment depth data OD1 and the automatically adjusted intensity E, E1, and E2 corresponding to the ultrasound intensity data OD2.

Therefore, the ultrasound generating device 100 according to the present disclosure may automatically adjust and irradiate ultrasound for each treatment region depth of the skin S using the previously measured information when the treatment is performed, so that ultrasound may be accurately irradiated to the irradiation position of the ultrasound, thereby improving the accuracy of the treatment.

In addition, the ultrasound generating device 100 according to the present disclosure may control the ultrasound to the optimal condition for each region A with one treatment process, so that the treatment time may be reduced and the treatment effect may be maximized.

When the treatment is completed for the skin layer of the corresponding region A with the corresponding treatment condition, the processor 122 may output the treatment completion state through the notification module 130. For example, the notification module 130 may be provided as at least one of a display module and a light-emitting diode for visual notification, and may be provided as a speaker for auditory notification.

That is, in the case that the treatment is completed with at least one of the treatment depth h1 or the ultrasound intensity (either one of E1, E2, and E3) which are the corresponding treatment conditions at the irradiation positions P1-1 to P1-n according to the first region, the processor 122 may output the treatment completion state through the notification module 130.

In addition, in the case that the treatment is completed with at least one of the treatment depth h2 or the ultrasound intensity (either one of E1, E2, and E3) which are the corresponding treatment conditions at the irradiation positions P2-1 to P2-n according to the second region, the processor 122 may output the treatment completion state through the notification module 130.

The communication module 112 of the ultrasound generating device 100 according to the present disclosure may further receive an image before the treatment and an image after the treatment for each of the plurality of regions. The image before the treatment and the image after the treatment for each of the plurality of regions may be obtained through the AI camera. The communication module 112 may receive and obtain image before the treatment and the image after the treatment for each of the plurality of regions while being connected to the external skin measuring device 200 through wired or wireless communication. The communication module 112 may also receive and obtain image before the treatment and the image after the treatment for each of the plurality of regions from a separate device or another server.

Here, the processor 122 may further transmit data for the image data before treatment, the image data after treatment, and the mapping information to the server 300. The server 300 may store the data for the image data before treatment, the image data after treatment, and the mapping information by creating a database.

The users (doctors) of other terminals that share treatment condition information through the server 300 may utilize the data for the image data before treatment, the image data after treatment, and the mapping information that are created in the database during treatment.

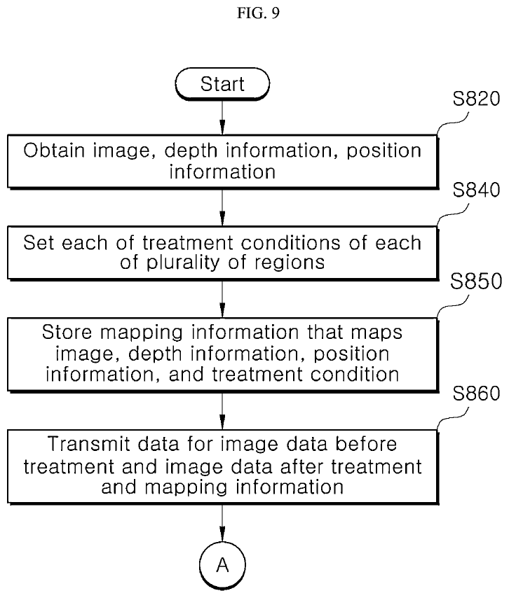

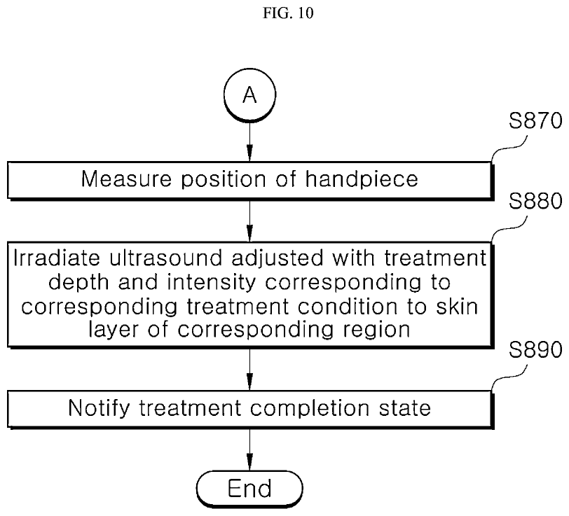

FIGS. 9 and 10 are flowcharts illustrating an example of a method for automatically adjusting ultrasound according to a depth of a skin treatment region of an ultrasound generating device according to the present disclosure.

Referring to FIG. 9 and FIG. 10, the method for automatically adjusting ultrasound may include an obtaining step S820, a treatment condition setting step S840, a mapping information storing step S850, and an ultrasound irradiating step S880. The obtaining step S820 may be operated in a state where the patient is fixed so that there is no movement of the patient.

The obtaining step may receive and obtain depth information on the skin layer of each of the plurality of regions A and the position information B of each of the plurality of regions A from the external skin measuring device 200 through the communication module 112. The depth information may be obtained through the first measurement module 212, and the position information B may be obtained through at least one of the navigation sensor, the position recognition camera, or the AI camera of the second measurement module 114. In the case that the second measurement module 114 is the AI camera, the position information B may further include image information including the plurality of regions A on the skin S. In addition, the communication module 112 may further receive the angle of the skin measuring device 200. The angle may be obtained through the first angle measurement module 212a. Here, the communication module 112 may receive and obtain the depth information and the position information B from a separate device or another server, and may also receive and obtain angle information.

The treatment condition setting step may set each of the treatment conditions of each of the plurality of regions A through the processor 122 (step S840). The memory 121 may store the treatment condition set respectively.

For example, the treatment condition is related to the treatment purpose, and may be for increasing fat, for reducing fat, for adding elasticity to the dermis or improving skin texture, for treating wrinkles or acne, for reducing pain, for lifting the jaw line in the case of removing fat from the cheek, and for lifting or tightening the skin. In addition, the treatment purpose may be for treating each region for skin improvement.

For another example, the treatment condition is for the target irradiation condition of ultrasound according to the treatment depth of each region, and may be to irradiate ultrasound with a specific temperature and specific energy the region such as forehead, lower jaw, nasolabial folds, around the eyes, nose, upper arms, and the like at the treatment depth of the nipple layer, upper dermis, lower dermis, upper fat, fat layer, SMAS, fascia, and the like. In addition, the target irradiation condition of ultrasound according to the treatment depth of each region may be to irradiate ultrasound with the target condition while varying the treatment depth of each region for skin improvement.

The mapping information storing step may store the mapping information that maps each of an image including the plurality of regions A on the skin S, the depth information on the skin layer of each of the plurality of regions A, the position information B of each of the plurality of regions A to a preset treatment condition through the memory 121 (step S850). The memory 121 may store the mapping information in which the entire position coordinate values (xn, yn) corresponding to each irradiation position for plurality of regions A, the depth values (zn) of the entire skin layer for the entire position coordinate values (xn, yn), and the treatment conditions in the entire position coordinates are mapped. The processor 122 may map the entire position coordinate values (xn, yn) corresponding to each irradiation position for plurality of regions A, the depth values (zn) of the entire skin layer for the entire position coordinate values (xn, yn), and the treatment conditions in the entire position coordinates.

The server 300 electrically connected to the controller 120 may learn the metadata, such as the position data ID1, the depth data ID2 of the skin layer, and the treatment condition data ID3, based on the treatment recommendation model M, and output the treatment depth data OD1 and the ultrasound intensity data OD2 of the recommended ultrasonic generator 110.

The treatment recommendation model M may be constructed to learn through the correlation between the position data ID1, the skin layer depth data ID2, and the treatment condition data ID3 included in the input data. The server 300 may transmit the treatment depth data OD1 and the ultrasound intensity data OD2 of the recommended ultrasonic generator 110 to the memory 121 of the controller 120. The memory 121 may store the received treatment depth data OD1 and the ultrasound energy data OD2 of the ultrasonic generator 110.

In the ultrasound irradiating step, when the ultrasonic generator 110 irradiates ultrasound to each of the plurality of regions A, the processor 122 may adjust ultrasound to be irradiated to the skin layer of the corresponding region A at least one of the treatment depth or intensity corresponding to the corresponding treatment condition (step S880). The ultrasonic generator 110 may irradiate at least one of the treatment depth or intensity of the adjusted ultrasound corresponding to the corresponding treatment condition to the skin layer of the corresponding region A. The processor 122 may control at least one of the angle or treatment direction of the ultrasonic generator 110 so that the ultrasound is accurately irradiated to the skin layer of the corresponding region A at least one of the treatment depth or intensity corresponding to the corresponding treatment condition based on at least one of the angle information or the treatment direction information stored in the memory 121.

For example, referring to FIG. 7, when an ultrasonic generator 110 having a transducer 111 sequentially irradiates ultrasound to the irradiation position P1-1 to P1-n according to the first region corresponding to the position coordinate values [(x1, y1), (x2, y1), . . . (xn, y1)] and skin depth values (z1, z2, . . . , zn) of the corresponding region A by the movement of the transfer unit 140, ultrasound may be irradiated with at least one of the automatically adjusted treatment depth h1 corresponding to the treatment depth data OD1 or the automatically adjusted intensity E, E1, and E2 corresponding to the ultrasound intensity data OD2. Here, ‘E’ may be ultrasound having the same intensity, and E1 and E2 may be ultrasound having different intensities. The ultrasonic generator 110 may irradiate ultrasound with the same intensity E to the irradiation positions P1-1 to P1-n according to the first region, and may irradiate ultrasound with different intensities E1 and E2. For example, the first region is a ball, and when the ultrasonic generator 110 sequentially irradiates ultrasound to the irradiation positions P1-1 to P1-n of the ball to remove fat from the ball, the ultrasonic generator may irradiate ultrasound with the same intensity E to the irradiation positions P1-1 to P1-n of the ball, and may irradiate ultrasound with different intensities E1 and E2.

As another example, referring to FIG. 8, when the ultrasonic generator 110 sequentially irradiates ultrasound to the irradiation positions P1-1 to P1-n according to the first region by the movement of the transfer unit 140 and then to the irradiation positions P2-1 to P2-n according to the second region corresponding to the position coordinate values [(x100, y2), (x100, y3), . . . (x100, yn)] and the skin depth values z2, z3, . . . , zn of the subsequent region A, the ultrasound may be irradiated with at least one of the automatically adjusted treatment depth h2 corresponding to the treatment depth data OD1 or the automatically adjusted intensity E, E1, and E2 corresponding to the ultrasound intensity data OD2. Here, ‘E’ may be ultrasound having the same intensity, and E1 and E2 may be ultrasound having different intensities. The ultrasonic generator 110 may irradiate ultrasound with the same intensity E to the irradiation positions P2-1 to P2-n according to the second region, and may irradiate ultrasound with different intensities E1 and E2. For example, the second region is the jaw border, and when the ultrasonic generator 110 sequentially irradiates ultrasound to the irradiation positions P2-1 to P2-n of the jaw border to lift the jaw border if the fat of the cheek is removed, the ultrasound may be irradiated with the same intensity E to the irradiation positions P2-1 to P2-n of the jaw border, and may irradiate ultrasound with different intensities E1, E2. The treatment depth of h2 may be deeper than the treatment depth of h1.

Meanwhile, the automatic ultrasound control method according to the present disclosure may further include a handpiece position measuring step (step S870).

The handpiece position measuring step may further measure the position of the ultrasonic generator 110 through the second measurement module 114. The second measurement module 114 may also measure the position of the handpiece 115 coupled with the cartridge housing in which the ultrasonic generator 110 is provided (step S870).

The ultrasound irradiation step may further control the ultrasound so that, when the ultrasonic generator 110 irradiates ultrasound for each of the plurality of regions A, the processor 122 identifies the mapping information corresponding to the position of the handpiece 115, and based on the mapping information, ultrasound is irradiated to the skin layer of the corresponding region A with at least one of the treatment depth or intensity corresponding to the corresponding treatment condition (step S880). The processor 122 may also control at least one of the treatment depth or intensity of ultrasound corresponding to the corresponding treatment condition to the skin layer of the corresponding region A based on the treatment depth data and ultrasound intensity data of the ultrasonic generator 110 stored in the memory 121. The ultrasonic generator 110 may irradiate at least one of the treatment depth or intensity of the adjusted ultrasound corresponding to the treatment condition to the skin layer of the corresponding region A.

Meanwhile, the automatic control method of ultrasound according to the present disclosure may further include a data transmitting step S860 and a notifying step S890.

The data transmitting step S860 may be performed after the mapping storage step S850. The data transmitting step S860 may be performed in synchronization with the ultrasound irradiating step S880, or may be performed after the ultrasound irradiating step S880. The data transmitting step S860 may be performed in synchronization with the notifying step S890, or may be performed after the notifying step S890.

The data transmitting step may receive the image before treatment and the image after treatment for plurality of regions through the communication module 112. The image before treatment and the image after treatment for the plurality of regions may be obtained through the AI camera. The communication module 112 may receive and obtain the image before treatment and the image after treatment for the plurality of regions while connected to the external skin measuring device 200 via wired or wireless communication. The communication module 112 may also receive and obtain the image before treatment and the image after treatment for plurality of regions from a separate device or another server. Here, the processor 122 may transmit data for the image data before treatment and the image data after treatment and the mapping information to the server 300 (step S860). The server 300 may store data for the image data before treatment and the image data after treatment and the mapping information in a database.

The users (doctors) of other terminals that share the treatment condition information through the server 300 may utilize data for the image data before treatment and the image data after treatment and the mapping information that have been stored in a database during the treatment.

The notifying step S890 may be performed after the ultrasound irradiating step S880. In the notifying step, when treatment is completed with the corresponding treatment condition on the skin layer of the corresponding region A through the processor 122, the treatment completion state may be notified through the notification module 130 (step S890). For example, the notification module 130 may be provided as at least one of a display module, a light-emitting diode, and a UI for visually notifying, and may be provided as a speaker for auditorily notifying.

That is, when the processor 122 completes the treatment with at least one of the treatment conditions, that is, the treatment depth h1 and the ultrasound intensity any one of E1, E2, and E3 at the irradiation positions P1-1 to P1-n according to the first region, or the treatment conditions, that is, the treatment depth h2 and the ultrasound intensity any one of E1, E2, and E3 at the irradiation positions P2-1 to P2-n according to the second region, the notification module 130 may notify the treatment completion state.

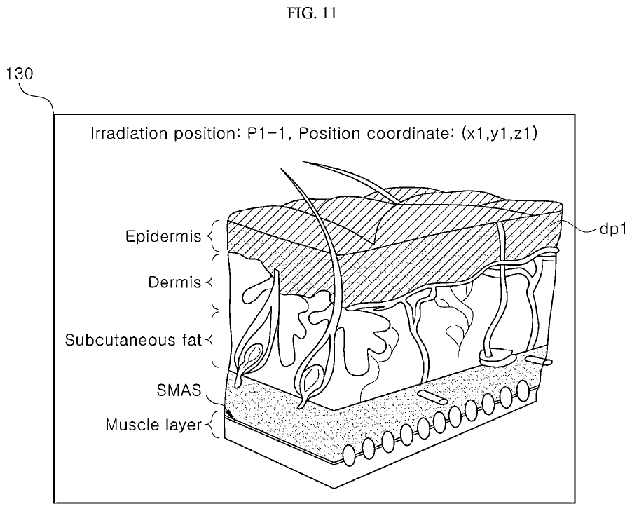

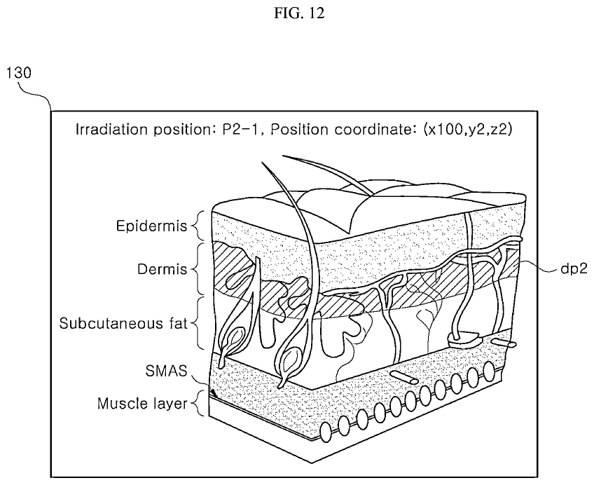

FIG. 11 is a diagram illustrating an example of displaying a treatment completion state according to a treatment depth at a current irradiation position corresponding to the epidermis as a UI through the notification module of FIG. 2. FIG. 12 is a diagram illustrating an example of displaying a treatment completion state according to a treatment depth at a current irradiation position corresponding to the dermis as a UI through the notification module of FIG. 2.

Referring to FIGS. 11 and 12, the notification module 130 may be provided as a UI screen on the hand piece. The UI screen may display the treatment completion state according to the treatment depth h1 at the current irradiation position P1-1 corresponding to the epidermis or the treatment depth h2 at the current irradiation position P2-1 corresponding to the dermis. The treatment depth dp1 corresponding to the epidermis or the treatment depth dp2 corresponding to the dermis may be displayed in a shade or a specified color. In addition, the UI screen may display the position coordinate (x1, y1, z1) at the current irradiation position P1-1 corresponding to the epidermis or the position coordinate (x100, y2, z2) at the current irradiation position P2-1 corresponding to the dermis.

Meanwhile, the ultrasound generating device 100 provided with automatic control function for ultrasound may irradiate ultrasound that is automatically controlled for each skin layer. Here, the skin layer may include the epidermis, the dermis, the subcutaneous fat, the muscle layer, the SMAS, and the like.

Meanwhile, the present disclosure may be provided as a computer program. The program may be stored in a computer-readable recording medium to execute a method performed by a computer.

The program may receive a photographed image including the plurality of regions A on the skin S, receive the depth Z of a skin layer measured for each of the plurality of regions A, and receive the position measured for each of the plurality of regions A.

The program may set the treatment condition for each of the plurality of regions A, and store the mapped position, the depth Z of the skin layer, and the treatment condition received for each of the plurality of regions A as the mapping information.

The program may provide a control signal to the ultrasonic generator 110 so that ultrasound is irradiated to the skin layer of the corresponding region A with at least one of a treatment depth h1 and h2 or intensity any one of E1, E2, and E3 corresponding to the corresponding treatment condition based on the mapping information.

At least one component may be added or deleted in accordance with the performance of the components illustrated in FIGS. 1 and 2. In addition, it will be readily understood by those skilled in the art that the mutual positions of the components may be changed in accordance with the performance or structure of the system.

Although FIGS. 9 and 10 describe the execution of multiple steps sequentially, this is merely an example of the technical idea of the present embodiment, and those skilled in the art to which the present embodiment pertains may modify and change the order described in FIGS. 9 and 10 without departing from principles of the present embodiment, or may execute one or more of the multiple steps in parallel, thereby allowing for various modifications and variations. Therefore, FIGS. 9 and 10 are not limited to a chronological order.

Meanwhile, the disclosed embodiments may be implemented in the form of a recording medium storing instructions executable by a computer. The instructions may be stored in the form of program codes, and when executed by a processor, may generate a program module to perform the operations of the disclosed embodiments. The recording medium may be implemented as a computer-readable recording medium.

The computer-readable recording medium includes all types of recording media storing instructions that may be deciphered by a computer. For example, there may be a ROM, a RAM, a magnetic tape, a magnetic disk, a flash memory, an optical data storage device, and the like.

The disclosed embodiments have been described with reference to the attached drawings as described above. A person skilled in the art to which the present disclosure pertains will understand that the present disclosure may be implemented in a different form from the disclosed embodiments without changing the technical idea or departing from principles of the present disclosure. The disclosed embodiments are illustrative and should not be construed as limiting.

According to the present disclosure, ultrasound can be accurately irradiated to the irradiation position of the ultrasound, thereby providing an effect capable of improving the accuracy of the treatment.

In addition, according to the present disclosure, since ultrasound can be irradiated by adjusting the optimal condition for each region in a single treatment, it provides an effect capable of maximizing the effect of the treatment while reducing the treatment time.

The effects of the present disclosure are not limited to the effects mentioned above, and other effects not mentioned will be clearly understood by those skilled in the art from the description.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An ultrasound generating device having an automatic ultrasound control function, the device comprising:
an ultrasonic generator configured to irradiate ultrasound to the skin;
a receiver configured to receive depth information for each of a plurality of skin regions from a skin measuring device, wherein the depth information for a respective skin region includes a plurality of depths corresponding to a plurality of skin layers for the respective skin region;
a memory configured to store mapping information, wherein the mapping information includes a correspondence between (A) a treatment condition and (B) position information of one or more skin regions to be treated for the treatment condition and depth information for each of the one or more skin regions to be treated for the treatment condition; and
a processor configured to control the ultrasonic generator based on the mapping information to irradiate ultrasound to each of the one or more skin regions to be treated for the treatment condition at respective depth(s) corresponding to the depth information.

2. The device according to claim 1, wherein the position information is obtained through at least one of a navigation sensor or a camera.

3. The device according to claim 2, wherein the position information is obtained from a patient while the patient is in a state where the patient is fixed so that there is no movement of the patient.

4. The device according to claim 1, wherein the position information is obtained through an artificial intelligence (AI) camera;
wherein the position information further includes image information including the plurality of skin regions.

5. The device according to claim 1, wherein the receiver is further configured to receive angle information of the skin measuring device.

6. The device according to claim 5, wherein the processor is further configured to determine whether the received angle information matches measured angle information corresponding to an angle of the ultrasonic generator.

7. The device according to claim 1, wherein controlling the ultrasonic generator includes checking for respective mapping information which corresponds to a respective position of a handpiece.

8. The device according to claim 1, wherein the processor is further configured to output a treatment completion state based on treatment being completed for a respective skin region.

9. The device according to claim 1, wherein the receiver is further configured to receive images of each of the plurality of skin regions from before and after a skin treatment procedure.

10. A device, comprising:
a skin measuring device configured to measure a depth of a skin layer; and
a transmitter configured to transmit the measured depth to an ultrasound generating device for mapping of a treatment condition to depth information for each of a plurality of skin regions.

11. The device according to claim 10, wherein the skin measuring device is configured to measure an angle of the skin measuring device; and
wherein the transmitter is further configured to transmit the measured angle to the ultrasound generating device.

12. An ultrasound generation system having an automatic ultrasound control function, comprising:
a skin measuring device configured to measure depth information for each of a plurality of skin regions, wherein the depth information for a respective skin region includes a plurality of depths corresponding to a plurality of skin layers for the respective skin region; and
an ultrasonic generator device configured to irradiate ultrasound to each of one or more skin regions to be treated for a treatment condition at respective depth(s) corresponding to the depth information based on mapping information, wherein the mapping information includes a correspondence between (A) the treatment condition and (B) position information of one or more skin regions to be treated for the treatment condition and depth information for each of the one or more skin regions to be treated for the treatment condition.

13. The device according to claim 12, wherein the ultrasonic generator device comprises:
an ultrasonic generator configured to irradiate ultrasound;
a receiver configured to receive the depth information;
a memory configured to store the mapping information; and
a processor configured to control the ultrasonic generator.
